(19) United States
(12) Patent Application Publication
(10) Pub. No.: US 2016/0074062 A1
(43) Pub. Date: Mar. 17, 2016

(54) DEVICE AND METHOD FOR HOLDING A NEEDLE ALONG A DESIRED PATH INTO THE BODY OF A PATIENT

(71) Applicant: Webb Medical LLC, Philadelphia, PA (US)

(72) Inventors: Steven B. Krupnick, Philadelphia, PA (US); David Krupnick, New York, NY (US); Anne Marie Cahill, Philadelphia, PA (US); Ganesh Krishnamurthy, Bryn Mawr, PA (US)

(21) Appl. No.: 14/849,734

(22) Filed: Sep. 10, 2015

Related U.S. Application Data
(60) Provisional application No. 62/051,540, filed on Sep. 17, 2014.

(51) Int. Cl. A61B 17/34

(52) U.S. Cl. CPC A61B 17/3403; A61B 2010/045

(57) ABSTRACT

A device and method for holding at needle along a desired percutaneous path into the body of a patient is disclosed. The device includes a base, a needle guide, a hinge, and a shapeable mass. The base has a top surface and a bottom surface. The needle guide has an inner surface and an outer surface, with the inner surface defining an elongate linear channel. The needle guide is pivotably connected to the base by the hinge, so that it can be oriented and the device positioned on the skin of the patient, whereupon a portion of the needle is disposed within the needle guide at its desired orientation. The shapeable mass is arranged to be secured between the outer surface of the needle guide and the top surface of the base to hold the needle guide in that desired orientation.

### CROSS-REFERENCE TO RELATED APPLICATIONS

This utility application claims the benefit under 35 U.S.C. §119(e) of Provisional Application Ser. No. 62/051,540 filed on Sep. 17, 2014, entitled Device And Method For Holding A Needle Along A Desired Path Into The Body Of A Patient. The entire disclosure of that provisional application is incorporated by reference herein.

### FIELD OF THE INVENTION

This invention relates generally to medical devices and more particularly to a device and method for holding a needle in a desired percutaneous path into the body of a patient.

### BACKGROUND OF THE INVENTION

Doing a needle biopsy on children is challenging. In this regard it is a common practice to insert a long, very thin needle a relatively short distance, e.g., one inch, into the child to establish the desired path along which the biopsy needle will be later introduced. This needle path determination step is accomplished by viewing the position of the thin needle using any suitable imaging technique, e.g., CT imaging. Owing to the fact that the needle is thin and only inserted a short distance into the child, it may become assume an undesired orientation or may even fall out of the patient during the imaging procedure. Thus, a need exists for a simple, low-cost, yet effective device which will hold a thin needle in position during some imaging procedure to establish a path for a later-to-be-introduced biopsy needle. The subject invention addresses that need.

### SUMMARY OF THE INVENTION

In accordance with one aspect of this invention there is provided a device for holding a needle that has been inserted into a patient along a desired percutaneous path. The device comprises a base, a needle guide, and an orientation fixation member. The base has a top surface and a bottom surface. The bottom surface is configured to be releasably secured to the skin of the patient adjacent the location of the needle. The needle guide has a surface configured to define an elongate linear channel having a longitudinal axis, whereupon a portion of the needle can be received within the needle guide extending along the longitudinal axis at a desired angle with respect to the base. The orientation fixing member comprises a shapeable mass configured to be secured between the needle guide and the top surface of the base to hold the needle guide in the desired orientation.

In accordance with one preferred aspect of this invention the device additionally comprises a hinge and wherein the needle guide has an inner surface and an outer surface. The inner surface is configured to define the elongate linear channel. The needle guide is pivotably connected to the base by the hinge, so that said needle guide can be oriented with respect to the base to the desired orientation. The shapeable mass is configured to be secured between the outer surface of the needle guide and the top surface of the base to hold the needle guide in the desired orientation.

In accordance with another preferred aspect of this invention the base is a generally planar member, but is conformable to conform to the contour of the skin the patient adjacent the location of the desired percutaneous path and the bottom surface of the base includes a releasably securable adhesive thereon to releasably secure the base to the skin of the patient.

In accordance with another preferred aspect of this invention a liner is provided to cover and protect the releasably securable adhesive until it is ready for use.

In accordance with another preferred aspect of this invention the shapeable mass is a putty-like substance.

In accordance with another preferred aspect of this invention the putty-like substance has an impermeable flexible coating, e.g., rubber or silicone, thereon.

In accordance with another preferred aspect of this invention the device is in the form of a kit housed in a sealed package, with the package being arranged to be opened to provide access to the integrally molded unit and the orientation fixing member.

In accordance with another aspect of this invention there is provided a method of holding a needle along a desired percutaneous path into the body of a patient. That method basically comprises providing a device comprising a base, a needle guide, a hinge, and an orientation fixation member. The base has a top surface and a bottom surface. The needle guide has an inner surface and an outer surface. The needle guide is pivotably connected to the base by the hinge. The orientation fixing member comprises a shapeable mass. The device is used by arranging the inner surface of the needle guide to define an elongate linear channel having a longitudinal axis. The needle guide is oriented with respect to the base to a desired orientation and the device is positioned so that the base is on the skin of the patient at a desired position, whereupon a portion of the needle is received within the needle guide and extends along the longitudinal axis at a desired angle with respect to the base. The device is secured to the skin of the patient at the desired position. The shapeable mass is secured in position between the outer surface of the needle guide and the top surface of the base to hold the needle guide in the desired orientation.

In accordance with one preferred aspect of the method of this invention a strip of releasably securable adhesive is disposed on a portion of the bottom surface of the base, and the method additionally comprises causing the releasably securable adhesive to engage the skin of the patient to secure the device on the skin of the patient at the desired position.

In accordance with another preferred aspect of the method of this invention the portion of the base which includes the strip of releasably securable adhesive is folded away from the remainder of the bottom surface of the base prior to the positioning of the base on the skin of the patient at the desired location.

In accordance with another preferred aspect of the method of this invention the portion of the base which includes the strip of releasably securable adhesive is folded towards the remainder of the bottom surface of the base after the positioning of the base on the skin of the patient at the desired location to thereby cause the releasably securable adhesive to engage the skin of the patient to secure the device to the skin of the patient at the desired position.

### DESCRIPTION OF THE DRAWING

FIG. 1 is a side elevation view of one exemplary embodiment of a device for holding a needle along an exemplary desired percutaneous path at an exemplary anterior-to-posterior angle, into the body of a patient, e.g., a child, to establish a path for a biopsy needle;

FIG. 2 is a front elevation view of the device taken along line 2-2 of FIG. 1;

FIG. 3 is a reduced size top plan view of a portion of the device shown in FIG. 1;

FIG. 4 is an enlarged sectional view taken along line 4-4 of FIG. 3;

FIG. 5 is reduced side elevation view similar to FIG. 1, but showing the device at a larger exemplary anterior-to-posterior angle than that shown in FIG. 1;

FIG. 6 is reduced side elevation view similar to FIG. 5, but showing the device at a smaller exemplary anterior-to-posterior angle than that shown in FIG. 1;

FIG. 7 is a front elevation view similar to FIG. 2, but showing the device at an exemplary lateral or side angle into the body of a patient;

FIG. 8 is a front elevation view similar to FIG. 2, but showing the device at another exemplary lateral or side angle into the body of a patient; and

FIG. 9 is a top plan view of a packaged kit containing the device 20.

### DETAILED DESCRIPTION OF THE PREFERRED EMBODIMENT

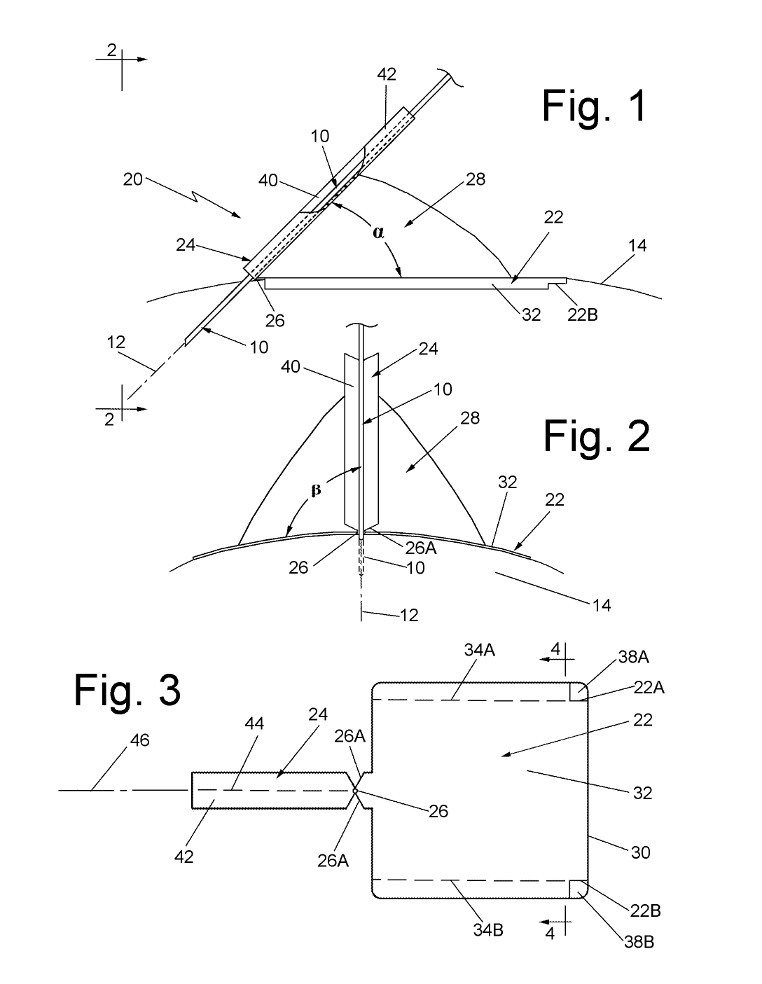

Referring now to the various figures of the drawing wherein like reference characters refer to like parts, there is shown at 20 in FIG. 1 one exemplary embodiment of a device for holding a needle 10 in a desired orientation along a desired percutaneous path 12 into the body 14 of a patient to establish a path for a biopsy needle (not shown).

The device 20 basically comprises a base 22, a needle guide 24, a hinge 26, and an orientation fixation member 28. The base, needle guide and hinge are in the form of a thin, planar, unitary member 30 (FIG. 3), which can be formed of any suitable card stock paper, a non-woven material or plastic. If paper stock, it can be 8 point to 14 point or 20 lb. to 140 lb. paper. If plastic, it can be a film of polyester, polyvinylchloride, or other similar materials. If a non-woven, it can be nylon, Tyvek® spun-bonded olefin, or other similar materials.

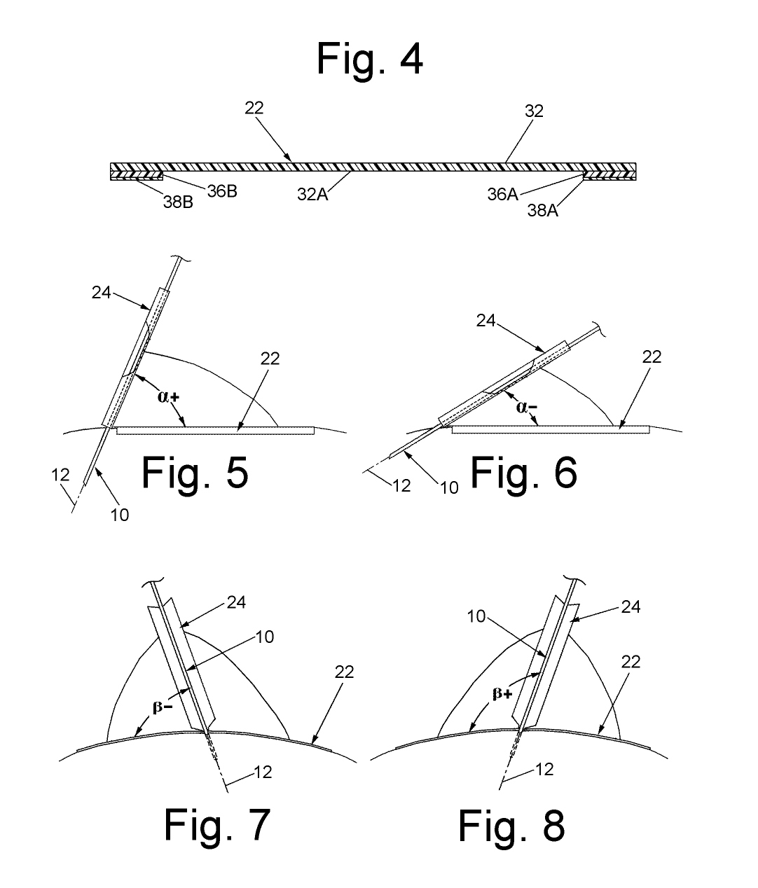

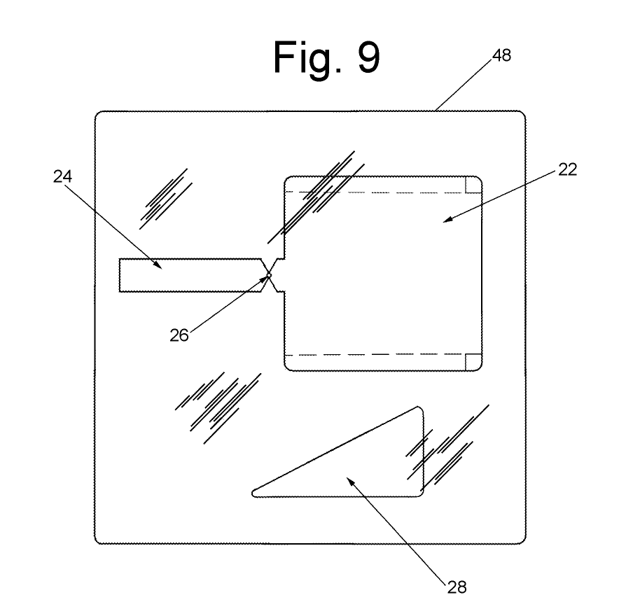

The unitary member 30 forming the base and needle guide is thin and sufficiently flexible so that the base 22 can be bent or curved into a shape conforming to the curvature of the portion of the body of the patient on which it is to be disposed. The base 22 of member 30 has a top surface 32 and a bottom surface 34 (FIG. 4). The bottom surface 34 of the base is arranged to be releasably secured to the skin of the patient 14. To that end, as best seen in FIG. 4, a strip 36A of a releasably securable adhesive is located on the bottom surface 32A of the base 22 adjacent on side edge of the base. A similar strip 36B of a releasably securable adhesive is located on the bottom surface of the base adjacent the opposite side edge of the base. The strip 36A extends from one side edge of the base up to a foldable line 34A provided in the base and extending along that side edge of the base. The strip 36B extends from the opposite side edge of the base up to a foldable line 34B provided in the base and extending along that oppose side edge of the base. The fold lines may be produced by scoring the material making up the base. The portions of the base between each fold line and its adjacent, respective side edge form a pair of marginal flaps that can be folded upward along the fold lines out of the plane of the remainder of the base to facilitate the placement of the base on the patient's skin as will be described later.

A protective, releasable cover or liner strip 38A is disposed over the undersurface of adhesive strip 36A to protect the adhesive until it is ready to be secured to the patient's skin, at which time the liner strip 36A is peeled off the adhesive strip to expose it, as will be described later. In a similar manner another releasable cover or liner strip 38B is disposed over the underside of the adhesive strip 36B.

The releasably securable adhesive can be any acrylic or rubber base, FDA approved medical grade skin contact adhesive. Preferably the adhesive is a “low ouch” adhesive, such as that sold by M&C Specialties Co., a division of ITW of 90 James Way, Southampton, Pa. 18966 along with the liner under the model designation #W542. A suitable low ouch adhesive is also available from Adchem Corporation 1852 Old Country Road Riverhead N.Y. 11901.

As best seen in FIGS. 1-3, the needle guide is an elongated rectangularly shaped extension of the member 30 having an inner surface 40 (FIG. 2) and an outer surface 42 (FIGS. 1 and 3). The needle guide 24 is pivotably connected to the base 22 via the hinge 26. The hinge 26 is located between a pair of V-shaped notches 26A and 26B in the extension. Those notches are provided to enable marginal edge portions of the needle guide to be folded together along a longitudinally extending fold line 44 so that the inner surface 40 of the needle guide forms an elongate linear channel of V-shaped cross-section. If desired, the fold line may be produced by scoring the material making up the unitary member 30. The elongate linear channel has a longitudinal axis 46 (FIG. 3) extending parallel to and immediately adjacent the fold line 40. The nadir of the V-shaped channel is arranged to receive the thin needle 10 to hold it in its desired orientation and position, as will be described later, to establish the desired path 12 for the biopsy needle.

As will be appreciated by those skilled in the art for a biopsy, the needle should be able to be adjusted to all angles, i.e., front-to-back (anterior-to-posterior) and side-to-side (lateral), in order to reach the target tissue safely inside the patient's body once needle entry has been performed. The hinge 26 is preferably constructed so as to enable the needle guide 24 to be pivoted omni-directionally, similarly to a ball joint, with respect to the base 22 to accommodate any needle orientation. For example, the joint 26 may be a living hinge. Alternatively, it may be in the form of a narrow strip of an elastic band, or a small body of silicone. In fact, it can be of any suitable construction providing that it enables the needle guide to be oriented to any anterior-to-posterior angle, while at the same time enabling the needle guide to be oriented to any lateral angle. FIGS. 1, 2 and 5-6 have been provided to show a few exemplary angulations of the needle guide with respect to the base. Thus, as can be seen in FIGS. 1 and 2 the needle guide is oriented so that the axis 12 extends at an acute anterior-to-posterior angle a with respect to the base, but is not at any lateral angle, i.e., the angle β with respect to the base is 90 degrees. In the exemplary orientation shown in FIG. 5, the needle guide is at a larger anterior-to-posterior angle, i.e., “α+”, than that shown in FIG. 1. In the exemplary orientation shown in FIG. 6, the needle guide is at a smaller anterior-to-posterior angle, i.e., “α−”, than that shown in FIG. 1. In the exemplary orientation shown in FIG. 7, the needle guide is at an exemplary lateral angle “β−”, which is less than the neutral (non-lateral) angle shown in FIG. 2. In the exemplary orientation shown in FIG. 8, the needle guide is at another exemplary lateral angle, i.e., “β+” which is greater than the neutral (non-lateral) angle shown in FIG. 2.

Use of the device 20 will typically be as follows. The thin needle will have been inserted into the patient by the user, e.g., a physician, and oriented to the desired orientation so that the distal end of the needle is directed along path 12 toward the situs of the tissue to be ultimately biopsied. Once the placement of the needle 10 has been accomplished needle guide 24 can to be bent out of the plane of the base 22 by pivoting it with respect to the base via the hinge 26 and folding the two marginal edge portions of the extension of the member 30 toward each other to form the V-shaped channel. After that has been accomplished (or before, if desired), the liner strips 38A and 38B can be removed, i.e., peeled off of their underlying adhesive strips, to thereby expose the adhesive of those strips. That can be accomplished by the user grasping the portion of the liner strip 38A located within the notch 22A to peel it off the adhesive strip 36A. The liner strip 38B can be removed by the user grasping a portion of that strip located within the notch 22B to peel it off of the adhesive strip 36B.

After that has been accomplished the marginal side edge portions of the base (i.e., the flaps) can then be folded upward along their respective fold lines 34A and 34B so that those portions, with the now exposed adhesive strips 36A and 36B are located above the plane of the undersurface 32A of the base located between the fold lines. The base 22 can then be slid or otherwise moved across the skin of the patient and the needle guide 24 pivoted omni-directionally about the hinge 26 until a portion of the needle 10 is located within the V-shaped channel, with the longitudinal axis of the needle coinciding with the axis 46 (nadir) of the V-shaped channel.

It should be pointed out at this juncture that if desired, the liner strip 38A can extend beyond the marginal rear edge and/or marginal side edge of the base adjacent the location of the notch 22A and the liner strip 38B can extend beyond the marginal rear edge and/or marginal side edge of the base adjacent the location of the notch 22B to facilitate the removal (peeling away) of the liner strips. In fact, it is contemplated that the device need not make use of notches in the base, if a portion of the liner strip extends beyond the marginal edge of the base at which it is located to provide a graspable or exposed portion which can be readily grasped by the user to remove the liner strip from the adhesive of the base.

Once the device 20 is in the desired position, the marginal side edge portions (i.e., flaps) of the base with the exposed adhesive strips can then be folded downward to cause the adhesive to engage the skin of the patient to thereby releasably secure the device 20 in place.

Once that has been accomplished the orientation fixation member 28 can be used to hold the needle guide 24, with the needle disposed therein at that precise angle. To that end, the orientation fixing member 26 comprises a shapeable mass that is arranged to be disposed on and secured between the outer surface 42 of the needle guide 24 and the top surface 32 of the base 22 to support and hold the needle guide at that angular orientation. As best seen in FIGS. 1 and 2 the shapeable mass 28 is of a generally wedge shape, but is conformable so that it can be readily adjusted to a shape having a precise angle to hold the V-shaped needle guide in the desired orientation. In particular, in accordance with one preferred aspect of this invention, the shapeable mass is putty-like, e.g., is formed of borosiloxane putty, such as that sold as LD-11 by Crazy Aaron Enterprises, Inc of Narberth, Pa. Other shapeable materials can be used in lieu of the exemplary putty-like material. If desired the base 22 may include a fold line, e.g., a linear scored portion (not shown), extending parallel to the back edge of the base to enable the portion of the base between the notches 22A and 22B to be folded upward to form a tray-like arrangement to facilitate the placement and holding of the orientation fixing member 28 on the base between it and the needle guide 24.

In accordance with a preferred aspect of this invention the device 20 (and any other embodiment of the device of this invention) is preferably provided in the form of a packaged kit, like shown in FIG. 5, so that the components making up the kit can be kept sterile until ready for use. To that end, the kit of components may be disposed in a sterile package 48. The package should be readily openable so that the unitary member 30 and the orientation fixing member 26 can be removed from the package and then assembled and used as described above.

It should be pointed out at this juncture that the exemplary embodiment of the device shown makes use of a base 22 that is square and is approximately 3 inches by 3 inches, with the extension forming the needle guide 24 being approximately 0.5 inch wide by 2.5 inches long. However, that exemplary embodiment is just that, i.e., exemplary. Thus, other shapes and sizes for the base and needle guide are contemplated in accordance with this invention. Moreover, the orientation fixing member need not be initially wedge shaped, but can be of any desired shape, provided that it can be modeled, shaped or otherwise adjusted to fit in between the bottom surface of the needle guide and the top surface of the base to hold the needle guide in the desired angular orientation with respect to the base.

As should be appreciated by those skilled in the art the subject invention can be used with both children and adults to stabilize a needle in various medical procedures, such as but not limited to, radiology needle stabilization procedures, endocrinology aspiration procedures, thyroid biopsy procedures, CT guided biopsy procedures, and targeted ablation procedures.

Without further elaboration the foregoing will so fully illustrate our invention that others may, by applying current or future knowledge, adopt the same for use under various conditions of service.

We claim:

1. A device for holding a needle that has been inserted into a patient along a desired percutaneous path, said device comprising a base, a needle guide, and an orientation fixation member, said base having a top surface and a bottom surface, said bottom surface being configured to be releasably secured to the skin of the patient adjacent the location of the needle, said needle guide having a surface configured to define an elongate linear channel having a longitudinal axis, whereupon a portion of said needle can be received within said needle guide extending along said longitudinal axis at a desired angle with respect to said base, said orientation fixing member comprising a shapeable mass configured to be secured between said needle guide and said top surface of said base to hold said needle guide in said desired orientation.

2. The device of claim 1 wherein said device additionally comprises a hinge and wherein said needle guide has an inner surface and an outer surface, said inner surface being configured to define said elongate linear channel, said needle guide being pivotably connected to said base by said hinge, so that said needle guide can be oriented with respect to said base to a desired orientation, said shapeable mass being configured to be secured between said outer surface of said needle guide and said top surface of said base to hold said needle guide in said desired orientation.

3. The device of claim 2 wherein said base is a generally planar member, but is conformable to conform to the contour of the skin the patient.

4. The device of claim 3 wherein said bottom surface of said base comprises a releasably securable adhesive to releasably secure said base to the skin of the patient.

5. The device of claim 2 wherein said channel is generally V-shaped in cross-section.

6. The device of claim 3 wherein said releasably securable adhesive has an undersurface and wherein said device additionally comprising a liner disposed on said undersurface of said adhesive to protect said adhesive until ready for use to secure said base to the skin of the patient.

7. The device of claim 6 wherein said adhesive is provided as a strip along a portion of said base adjacent an edge thereof, and wherein said strip is arranged to be folded upward along a fold line.

8. The device of claim 6 wherein said base includes a pair of said edges and wherein said adhesive is provided as a pair of strips, one of pair of said strips extending along a portion of said base adjacent one side edge of said pair of side edges and the other of said strips extending along a portion of said base adjacent one side edge of the other of said pair of side edges.

9. The device of claim 2 wherein said hinge is constructed to enable said needle guide to be oriented at any anterior-to-posterior angle and at any lateral angle.

10. The device of claim 2 wherein said base, said needle guide and said hinge comprise an integral unit.

11. The device of claim 2 wherein said shapeable mass is putty-like so that it can be adjusted to a shape to hold said needle guide in said desired orientation.

12. The device of claim 11 wherein said shapeable mass is of a general wedge shape and is putty-like so that it can be adjusted to a shape to hold said needle guide in said desired orientation.

13. The device of claim 12 wherein said shapeable mass comprises borosiloxane putty.

14. The device of claim 2 wherein said device is in the form of a kit housed in a sealed package, said package being arranged to be opened to provide access to said base, said needle guide, said hinge, and said orientation fixing member.

15. The device of claim 1 wherein said shapeable mass comprises a putty-like substance having an impermeable flexible coating thereon.

16. The device of claim 15 wherein said flexible coating comprises rubber or silicone.

17. A method of holding a needle along a desired percutaneous path into the body of a patient, said method comprising:
a) providing a device comprising a base, a needle guide, a hinge, and an orientation fixation member, said base having a top surface and a bottom surface, said needle guide having an inner surface and an outer surface, said needle guide being pivotably connected to said base by said hinge, said orientation fixing member comprising a shapeable mass;
b) arranging said inner surface of said needle guide to define an elongate linear channel having a longitudinal axis;
c) orienting said needle guide with respect to said base to a desired orientation and positioning said base on the skin of the patient at a desired position whereupon a portion of said needle is received within said needle guide and extends along said longitudinal axis at a desired angle with respect to said base;
d) securing said device to the skin of the patient at said desired position; and
e) securing said shapeable mass between said outer surface of said needle guide and said top surface of said base to hold said needle guide in said desired orientation.

18. The method of claim 17 wherein a strip of releasably securable adhesive is disposed on a portion of said bottom surface of said base, and wherein said method additionally comprises:
e) causing said releasably securable adhesive to engage the skin of the patient to secure said device on the skin of the patient at said desired position.

19. The method of claim 19 additionally comprising:
f) folding said portion of said base which includes said strip of releasably securable adhesive away from the remainder of the bottom surface of said base prior to the positioning of said base on the skin of the patient at said desired location.

20. The method of claim 19 additionally comprising;
g) folding said portion of said base which includes said strip of releasably securable adhesive towards the remainder of the bottom surface of said base after the positioning of said base on the skin of the patient at said desired position to thereby cause said releasably securable adhesive to engage the skin of the patient to secure said device to the skin of the patient at said desired position.

21. The method of claim 17 wherein a liner strip is disposed over said strip of releasably securable adhesive to protect said releasably securable adhesive until it is ready for use.

22. The method of claim 17 wherein said hinge is constructed to enable said needle guide to be oriented at any anterior-to-posterior angle and at any lateral angle.

23. The method of claim 17 wherein said device is provided in the form of a kit housed in a sealed package, and wherein said method additionally comprises opening said package to provide access to said base, said needle guide, said hinge, and said orientation fixing member.
